(19) United States
(12) Patent Application Publication
(10) Pub. No.: US 2004/0132728 A1
(43) Pub. Date: Jul. 8, 2004

(54) Combinations of atorvastatin and alpha1adrenergic receptor antagonists

(75) Inventors: Nicholas Pullen, Sandwich (GB); Richard James Thurlow, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/663,082

(22) Filed: Sep. 16, 2003

Related U.S. Application Data
(60) Provisional application No. 60/417,521, filed on Oct. 9, 2002.

(30) Foreign Application Priority Data
Sep. 17, 2002 (GB) 0221579.6

(51) Int. Cl. A61K 31/5377; A61K 31/517; A61K 31/401

(52) U.S. Cl. 514/234.2; 514/423; 514/252.17; 514/266.22

(57) ABSTRACT

Combinations of atorvastatin and α₁ adrenergic receptor antagonists, the use of such combinations in the treatment of benign prostatic hyperplasia (BPH), methods of treating BPH using such combinations and medicaments containing such combinations are described.

This application claims priority to U.S. Provisional Application No. 60/417,521, filed Oct. 9, 2002, and GB Application No. 0221582.0, filed Sep. 17, 2002.

This invention relates to combinations of atorvastatin and α₁ adrenergic receptor antagonists, the use of such combinations in the treatment of benign prostatic hyperplasia (BPH), methods of treating BPH using such combinations and medicaments containing such combinations.

BPH is a chronically progressive disease that can lead to complications such as acute urinary retention, recurrent urinary tract infections, bladder stones and renal dysfunction. The prevalence and average severity of lower urinary tract symptoms (LUTS) associated with BPH in men increases with age.

BPH leads to an increase in prostate volume, creating urethral and bladder outflow obstruction as well as secondary changes in bladder function. The effects of this are manifested by both storage (irritative) and voiding (obstructive) symptoms, giving rise to nocturia, urinary urgency and poor urinary flow.

In patients with BPH, blockade of sympathetic (adrenergic) nerve innervations of the prostate reduces intra-urethral pressure by about 50% (J. Urol., 1982, 128, 836), alleviating the symptoms of outflow obstruction. In particular, adrenergic receptors of the α₁ subtype predominate in the prostate and lower urinary tract and α₁ adrenoceptor-specific antagonists have been identified which preferentially relax prostatic smooth muscle compared with cardiovascular smooth muscle. Clinical trials have confirmed this hypothesis and several α₁ antagonists such as tamsulosin, terazosin, alfuzosin and doxazosin are now marketed for the treatment of BPH.

Many reviews of α₁ adrenoceptor antagonists are available, for example see Prostate Cancer Prostatic Dis. 2000, 3, 76-83; Annu. Rep. Med. Chem. 2000, 35, 221-230; Expert Opin. Invest. Drugs, 1999, 8, 2073-2094; Prostate Cancer Prostatic Dis., 1999, 2, 110-119; J. Med. Chem., 1997, 40, 1293; Pharm. Res., 1996, 33, 145.

While, the introduction of pharmacological therapies has heralded some improvement in the impact of the symptoms and the need for surgical intervention for BPH, the overall effects are moderate and the needs of patients and physicians are still largely unmet. Also there is no evidence that the currently available pharmacological therapies are effective at controlling either the bladder hypertrophy, detrusor instability, or prostate/bladder fibrosis associated with BPH.

Atorvastatin calcium, disclosed in U.S. Pat. No. 5,273,995, is currently sold as Lipitor®, and is [R—(R*, R*)]-2-(4-fluorophenyl)-β,δ-dihydroxy-5-(1-methylethyl)-3-phenyl-4-[(phenylamino)carbonyl]-1H-pyrrole-1-heptanoic acid hemi calcium salt, represented by formula I below:

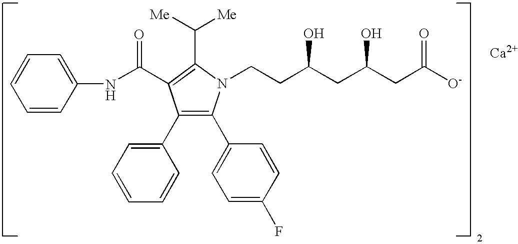

It is known to be a potent inhibitor of HMG-CoA reductase.

Italian Patent Application No. 048658 describes the use of HMG-CoA reductase inhibitors for the treatment of BPH. JP56115717 discloses the use of two HMG-CoA reductase inhibitors (monacholin K and ML-236B) for treating prostatomegaly. US2002/0004521 describes the use of atorvastatin for the treatment of BPH. Combinations of 5α-reductase inhibitors with α-adrenergic receptor antagonists are described for use in the treatment of BPH in U.S. Pat. No. 5,753,641. WO 99/11260 concerns the combination of atorvastatin with an antihypertensive agent, which may comprise an α-adrenergic receptor antagonist. Such combinations are useful in the treatment of angina pectoris, atherosclerosis, combined hypertension and hyperlipidemia and for the treatment of subjects presenting with symptoms of cardiac risk.

This invention provides the use of a combination of (A) atorvastatin or a pharmaceutically acceptable derivative thereof and (B) an α₁-adrenergic receptor antagonist or a pharmaceutically acceptable derivative thereof, in the manufacture of a medicament for the treatment of BPH. Pharmaceutically acceptable derivatives include pharmaceutically acceptable salts and pharmaceutically acceptable solvates.

Further, it provides the use of a combination of (A) and (B) for the treatment of BPH in order to improve lower urinary tract symptoms and urinary flow rates, limit progression of the disease and reverse the pathological changes in the bladder and prostate associated with the disease, thus reducing the incidence of urinary retention and the requirement for surgery.

The combinations of the invention may have the advantage that, due to a synergistic interaction between the active ingredients, they are more potent, have a longer duration of action, more effectively reduce disease progression and, therefore, the requirement for surgical intervention, have a broader range of activity, are more stable, have fewer side effects or are more selective (in particular they may have beneficial effects in BPH without causing undesirable cardiovascular effects) or have other more useful properties than the prior art.

In one embodiment, there is provided the use of a combination of (A) atorvastatin or a pharmaceutically acceptable derivative thereof and (B) an α₁-adrenergic receptor antagonist or a pharmaceutically acceptable derivative thereof, in the manufacture of a medicament for combination therapy by simultaneous, sequential or separate administration of (A) and (B) in the treatment of BPH. α₁-Adrenergic receptor antagonists useful for (B) include, but are not limited to, terazosin (U.S. Pat. No. 4,026,894), doxazosin (U.S. Pat. No. 4,188,390), prazosin (U.S. Pat. No. 3,511,836), bunazosin (U.S. Pat. No. 3,920,636), alfuzosin (U.S. Pat. No. 4,315,007), naftopidil (U.S. Pat. No. 3,997,666), tamsulosin (U.S. Pat. No. 4,703,063), silodosin (U.S. Pat. No. 5,387,603); the compounds disclosed in International Application No. PCT/IB03/00998, in particular 5-cyclopropyl-7-methoxy-2-(2-morpholin-4-ylmethyl-7,8-dihydro[1,6]-naphthyridin-6(5H)-yl)₄(3H)-quinazolinone (example 11), and 4-amino-6,7-dimethoxy-2-(5-methanesulfonamido-1,2,3,4-tetrahydroisoquinol-2-yl)-5-(2-pyridyl)quinazoline (International Patent Application Publication No. WO 98/30560, example 19); and pharmaceutically acceptable derivatives thereof. Preferred α-adrenergic receptor antagonists are doxazosin, 5-cyclopropyl-7-methoxy-2-(2-morpholin-4-ylmethyl-7,8-dihydro[1,6]-naphthyridin-6(5H)-yl)-4(3H)-quinazolinone and 4-Amino-6,7-dimethoxy-2-(5-methanesulfonamido-1,2,3,4-tetrahydroisoquinol-2-yl)-5-(2-pyridyl)quinazoline and pharmaceutically acceptable derivatives thereof. The mesylate salt of 4-Amino-6,7-dimethoxy-2-(5-methanesulfonamido-1,2,3,4-tetrahydroisoquinol-2-yl)-5-(2-pyridyl)quinazoline is of particular interest (see WO 01/64672).

One embodiment of the invention comprises a pharmaceutical composition comprising (A) atorvastatin or a pharmaceutically acceptable derivative thereof and (B) doxazosin or a pharmaceutically acceptable derivative thereof.

An alternative embodiment comprises a pharmaceutical composition comprising (A) atorvastatin or a pharmaceutically acceptable salt thereof and (B) 5-cyclopropyl-7-methoxy-2-(2-morpholin-4-ylmethyl-7,8-dihydro[1,6]-naphthyridin-6(5H)-yl)-4(3H)-quinazolinone or a pharmaceutically acceptable salt thereof.

A further embodiment comprises a pharmaceutical composition comprising (A) atorvastatin or a pharmaceutically acceptable derivative thereof and (B) 4-amino-6,7-dimethoxy-2-(5-methanesulfonamido-1,2,3,4-tetrahydroisoquinol-2-yl)-5-(2-pyridyl)quinazoline or a pharmaceutically acceptable derivative thereof.

An additional embodiment comprises a pharmaceutical composition comprising (A) atorvastatin or a pharmaceutically acceptable derivative thereof and (B) doxazosin or a pharmaceutically acceptable derivative thereof.

In a further embodiment there is provided a medicament containing, separately or together, (A) atorvastatin or a pharmaceutically acceptable derivative thereof and (B) 4-amino-6,7-dimethoxy-2-(5-methanesulfonamido-1, 2,3,4-tetrahydroisoquinol-2-yl)-5-(2-pyridyl)quinazoline or a pharmaceutically acceptable derivative thereof, for simultaneous, sequential or separate administration in the treatment of BPH.

In an alternative embodiment there is provided a medicament containing, separately or together, (A) atorvastatin or a pharmaceutically acceptable derivative thereof and (B) 5-cyclopropyl-7-methoxy-2-(2-morpholin-4-ylmethyl-7,8-dihydro[1,6]-naphthyridin-6(5H)-yl)-4(3H)-quinazolinone or a pharmaceutically acceptable salt thereof, for simultaneous, sequential or separate administration in the treatment of BPH.

A further embodiment comprises a medicament containing separately or together, (A) atorvastatin or a pharmaceutically acceptable derivative thereof and (B) doxazosin or a pharmaceutically acceptable salt thereof, for simultaneous, sequential or separate administration in the treatment of BPH.

In a further embodiment there is provided a pharmaceutical composition comprising a mixture of effective amounts of (A) as hereinbefore defined and (B) as hereinbefore defined, optionally together with a pharmaceutically acceptable carrier.

In the pharmaceutical compositions of the present invention, (A) is present in an amount ranging from 10 mg to 80 mg per dose, and (B) is present in an amount ranging from 0.1 mg to 20 mg per dose. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The pharmaceutical compositions of the present invention can be administered alone but will generally be administered in a mixture with a suitable pharmaceutical excipient, diluent or carried selected with regard to the intended route of administration and standard pharmaceutical practice. For example, the pharmaceutical compositions can be administered orally, buccally or sublingually in the form of tablets, capsules, multi-particulates, gels, films, ovules, elixirs, solutions or suspensions, which may contain flavouring or colouring agents, for immediate-, delayed-, modified-, sustained-, pulsed- or controlled-release applications. The pharmaceutical compositions may also be administered as fast-dispersing or fast-dissolving dosage forms or in the form of a high energy dispersion or as coated particles. Suitable formulations of the pharmaceutical compositions may be in coated or uncoated form.

Solid pharmaceutical compositions, for example tablets, may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

The pharmaceutical compositions may also be administered in the form of a suppository for rectal administration. These compositions can be prepared by mixing the drug with a suitable non-irritating excipients which is solid at ordinary temperatures but liquid at the rectal temperature and will therefore melt in the rectum to release the drug. Such materials are cocoa butter and polyethylene glycols.

The combination of this invention may also be administered in a controlled-release dosage formulation such as a slow release or a fast release formulation. Such controlled release formulations of the combination of this invention may be prepared according to methods well known to those skilled in the art.

Pharmaceutical compositions according to the invention may contain 0.1%-95% of the compounds of this invention, preferably 1%-70%.

Still further provided by the present invention is a method of treating BPH comprising administering to a subject in need of such treatment amounts of (A) as hereinbefore described and (B) as hereinbefore described which are together effective.

Yet further, there is provided by the present invention a pharmaceutical product containing (A) and (B) as hereinbefore defined, as a combined preparation for simultaneous, separate or sequential use in treating BPH.

“Effective amounts” as used herein is an amount of (A) and (B) that will elicit the biological or medical response being sought. The daily dose of (A) and (B) employed in the method of treatment is similar to the doses described for use in the pharmaceutical compositions hereinbefore described. In the method of treatment according to the present invention (A) and (B) can be administered together combined in a single dosage form, or they can be administered separately, essentially concurrently, each in its own dosage form but as part of the same therapeutic treatment program, and it is envisaged that (A) and (B) may be separately administered, at different times and by different routes.

The utility of the combination of the present invention as medical agents in the treatment of BPH is demonstrated by the activity of the combination in the protocol described below:

Efficacy of Atorvastatin and Doxazosin on Benign Prostate Hyperplasia (BPH) in the Spontaneous Hypertensive Rat (SHR).

This study is designed to investigate the effects of atorvastatin at 1, 10 and 30 mg/kg, and concomitant treatment with 0.1 mg/kg of doxazosin on bladder function (as assessed by cystometry i.e. the flow of urine through the bladder/urethra) and gross prostate morphology (prostate weight, stromal/epithelial volume) of spontaneous hypertensive rats (SHR).

SHR's have increased prostate size (increased stromal and epithelial growth), and bladder hyperactivity relative to their normotensive Wistar-Kyoto (WKY) counterparts. These changes in bladder function and prostate morphology reflect those observed in men with BPH. All tissue samples collected (prostates) were examined for size and gross cellular morphology (stromal content and epithelial thickness) as an indication of BPH.

The animals were group housed under 12:12 hour light:dark cycle. They were offered a standard rat diet and water ad libitum.

One hundred, 12 week old, spontaneously hypertensive male rats (SHR; Harlan UK) were allocated randomly to 5 treatment groups; (i) placebo (ii) combination at the rate of 1 mg/kg/d po, (iii) combination 10 mg/kg/d po (iv) combination 30 mg/kg/d po or (v) doxazosin 0.1 mg/kg/d. A further control group consisting of 20 normotensive rats Wistar-Kyoto (WKY) was included in the study, these animals received placebo treatment.

The SHR and WKY animals were administered orally their respective treatments for a period of 60 days. Micturition parameters (frequency, volume void and total volume over a 2 hour period) were assessed on a sub-group of animals on days 0, 25 and 50 of the study.

On days 30 and 60 of the experiment, 10 animals from each treatment group were selected randomly to undergo terminal anaesthesia studies to assess the effects of treatment on bladder/urethral function (cystometry, i.e. urine flow through the bladder/urethra) and prostate size.

Animals were anaesthetised using urethane (1.2 g/kg, i.p.). Depth of anaesthesia was assessed by the stability of blood pressure and heart rate, and by an absence of hind limb withdrawal in response to paw pinch. Supplementary doses of urethane (0.1 g kg-1, i.v.) were given where necessary. The trachea was intubated to maintain a patent airway. The left jugular vein was cannulated for drug administration, and the left common carotid artery was cannulated with a heparinised cannula (20 units/ml heparin in 0.9% w/v saline) for the measurement of arterial blood pressure and for sampling arterial blood for blood gas analysis.

Blood pressure was measured using a pressure transducer (Gould Statham P23 Db), and the heart rate (HR) derived electronically on-line from the blood pressure using PoneMah (Linton Pty Ltd UK). Body temperature was monitored with a rectal temperature probe and maintained between 36-38° C. using a homeothermic blanket system (Harvard, UK).

The animals either spontaneously breathed air or were artificially ventilated, and blood gases were maintained between 90-130 mmHg Po2, 40-50 mmHg PCo2 and pH 7.3-7.4. Adjustments of the supplemented oxygen levels (spontaneously breathing animals) and respiratory pump rate and volume (artificially ventilated animals) were made as necessary to maintain blood gas and pH balance.

The urinary bladder was exposed by a midline abdominal incision. A cut was made in the bladder dome and double lumen cannula (0.52 mm internal and 1.2 mm external diameter) was inserted into the bladder, one of which was connected to a pressure transducer (Gould Statham P23 Db) to record intravesical bladder pressure, and the other connected to a syringe pump for the infusion of saline (0.9% w/v) to evoke the micturition reflex. The rate (0.046 ml/min) of infusion of saline into the bladder was chosen to simulate the maximal hourly diuresis rate (Klevmark, 1974).

Following the surgical procedure the animals were allowed to stabilise for c. 30 min. After the stabilisation period, cystometry was performed. Bladder/urethral function and total volume voided were assessed over a 60 minute period.

A microsphere technique was employed to assess the effects of treatment on prostate and bladder blood flow (see Das et al, 2002). Briefly, 2 million Nuflow fluorescent red microspheres (IMT; 15 μm diameter suspended in 0.4 mL of 0.9% saline and 0.01% Tween-80) were injected via the carotid catheter. Blood samples were collected prior to, during and following the infusion of microspheres. Five minutes after the infusion the rats were euthanised and the bladder, urethra and prostate collected and blood flow determined. Any change in prostate size alters bladder blood flow and improves bladder function.

Following cystometry, a 2 mL blood sample was collected into heparinised tubes, plasma prepared as soon as possible and stored at −20° C. pending analysis for the combination.

Immediately following the termination of the experiment the prostate of the rats was collected, weighed, stored in 10% formalin pending gross morphological examination of the stroma and epithelial thickness.

Differences between treatment groups were examined using ANOVA.

Experimental:

5-Cyclopropyl-7-methoxy-2-(2-morpholin-4-ylmethyl-7,8-dihydro[1,6]-naphthyridin-6(5H)-yl)-4(3H)-quinazolinone (example 11 of International Application No. PCT/IB03/00998)

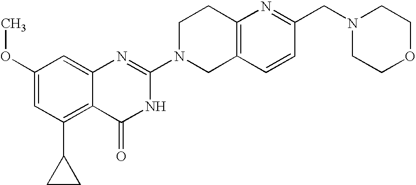

may be prepared via the following preparations:

### Preparation 1

### 2,6-Difluoro-N-(2-hydroxy-1,1-dimethylethyl)-4-methoxybenzamide

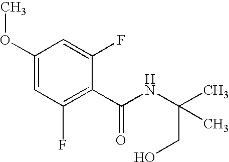

2,6-difluoro-4-methoxybenzoic acid (Mol. Cryst. Liq. Cryst. 1989; 172; 165) (2.09 g, 11.1 mmol) was suspended in dichloromethane (110 mL) and a few drops of N,N-dimethylformamide was added followed by oxalyl chloride (2.79 g, 22.2 mmol). The reaction mixture was stirred for 45 minutes at room temperature, after which time a clear homogeneous solution had formed. The reaction mixture was concentrated under reduced pressure and redissolved in dichloromethane (100 mL). The reaction mixture was then added slowly to an ice-cold solution of amino-2-methylpropanol (3.56 g, 40 mmol), in dichloromethane (50 mL). After stirring at room temperature for 1 hour, the reaction mixture was washed with water (75 mL), 0.2N hydrochloric acid (50 mL), dried (MgSO₄) and concentrated under reduced pressure to give the title compound as a white solid (2.77 g, 96%).

¹H-nmr (CDCl₃, 400 MHz) δ: 1.38 (s, 6H), 3.70 (m, 2H), 3.80 (s, 3H), 5.90 (bs, 1H), 6.42 (2×s, 2H).

LRMS: m/z (ES⁺) 260 [MH⁺]

### Preparation 2

### 2-(2,6-Difluoro-4-methoxyphenyl)-4,4-dimethyl-4,5-dihydro-1,3-oxazole

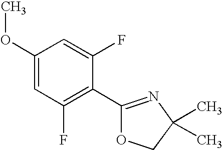

To a solution of the alcohol from preparation 1 (2.75 g, 10.6 mmol) in anhydrous dichloromethane (50 mL) was added thionyl chloride (1.43 g, 12 mmol) and the reaction stirred for 1.5 hours at room temperature. The reaction mixture was poured into 1 M sodium hydroxide solution (50 mL) and extracted with dichloromethane (2×50 mL). The combined organic solutions were dried (MgSO₄) and concentrated under reduced pressure. The residue was purified by column chromatography on silica gel (eluting with dichloromethane:methanol 96:4) to give the title compound as a clear oil (2.40 g, 94%).

¹H-nmr (CDCl₃, 400 MHz) δ: 1.40 (s, 6H), 3.80 (s, 3H), 4.04 (s, 2H), 6.42 (m, 2H).

LRMS: m/z (ES⁺) 242 [MH⁺]

### Preparation 3

### 2-(2-Cyclopropyl-6-fluoro-4-methoxyphenyl)-4,4-dimethyl-4,5-dihydro-1,3-oxazole

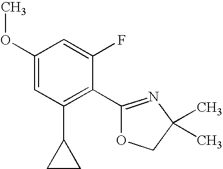

To a solution of cyclopropyl bromide (12.1 g, 100 mmol) in anhydrous tetrahydrofuran (100 mL) was added magnesium turnings (2.4 g, 100 mmol) followed by a crystal of iodine, at room temperature. After a few minutes the reaction initiated and came to reflux without any additional heating. When the reflux was complete the reaction was cooled to room temperature and stirred for 2 hours. A solution of the fluoro compound from preparation 2 (9.64 g, 40 mmol) in tetrahydrofuran (50 mL) was cooled in an ice-bath to 0° C., and the grignard solution (50 mL) was added dropwise over 15 minutes, the cooling bath was removed and reaction warmed to room temperature and stirred for 1 hour. Further grignard solution (20 mL) was added and stirred for 1 hour. Further grignard solution (10 mL) was added and stirred for 1 hour. The reaction mixture was then quenched with 1 M citric acid (30 mL), as some solid remained undissolved 2M hydrochloric acid (30 mL) was added. The resultant mixture was partitioned between ethyl acetate (400 mL) and water (200 mL), basified with concentrated ammonia solution and the organic layer separated, washed with water (150 mL), brine (150 mL), dried (MgSO₄) and concentrated under reduced pressure. The residue was purified by flash chromatography on silica gel (eluting with hexane:isopropyl alcohol 85:15) to give the title compound as a clear oil (10.32 g, 98%).

¹H-nmr (CDCl₃, 40 MHz) δ: 0.66 (m, 2H), 0.94 (m, 2H), 1.40 (s, 6H), 2.18 (m, 1H), 3.78 (s, 3H), 4.07 (s, 2H), 6.25 (s, 1H), 6.42 (m, 1H).

LRMS: m/z (ES⁺) 286 [MNa⁺]

### Preparation 4

### 2-Cyclopropyl-6-fluoro-4-methoxybenzonitrile

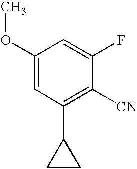

Pyridine (31.6 g, 400 mmol) was added to a solution of the compound from preparation 3 (10.32 g, 39.2 mmol) in ethyl acetate (150 mL), followed by phosphorous oxychloride (12.27 g, 80 mmol). The reaction was stirred at reflux for 5 hours, cooled and poured onto ice. This aqueous mixture was extracted with ethyl acetate, the organic solution washed with 2M hydrochloric acid, and brine then dried (MgSO₄) and evaporated under reduced pressure. The residue was purified by column chromatography on silica gel using dichloromethane to afford the title compound as a white solid, 6.41 g.

¹H-nmr (CDCl₃, 400 MHz) δ: 0.88 (m, 2H), 1.16 (m, 2H), 2.20 (m, 1H), 3.80 (s, 6H), 6.21 (s, 1H), 6.49 (m, 1H); LRMS: m/z (ES⁺) 214 [MNa⁺]

### Preparation 5

### 2-Amino-6-cyclopropyl-4-methoxybenzonitrile

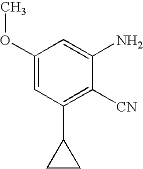

The fluoro compound from preparation 4 (3.0 g, 15.7 mmol) was added to a saturated solution of 0.88 ammonia in dimethylsulphoxide (20 mL), and the solution stirred in a sealed vessel for 18 hours at 150° C. The cooled mixture was partitioned between ethyl acetate and water and the layers separated. The organic phase was dried (MgSO₄) and evaporated under reduced pressure. The residue was purified by column chromatography on silica gel using dichloromethane as eluant to afford the title compound as a white crystalline solid, 1.28 g.

¹H-nmr (CDCl₃, 400 MHz) δ: 0.74 (m, 2H), 1.02 (m, 2H), 2.10 (m, 1H), 3.76 (s, 6H), 4.38 (bs, 2H), 5.82 (s, 1H), 6.00 (s, 1H).

LRMS: m/z (ES⁺) 211 [MNa⁺]

### Preparation 6

### 5-Cyclopropyl-7-methoxy-2,4(1H,3H)-quinazolinedione

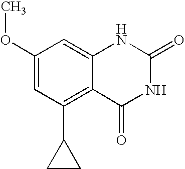

A solution of the compound from preparation 5 (1.25 g, 6.65 mmol) in N,N-dimethylformamide (10 mL) and 1,8-diazabicyclo[5.4.0]undec-7-ene (2 mL) was cooled to −78° C., and solid carbon dioxide added. The reaction vessel was sealed and heated to 140° C. for 18 hours. The cooled mixture was poured into water (150 mL), then acidified using 2N hydrochloric acid, and the mixture stirred for 10 minutes. The resulting precipitate was filtered off, washed with water and acetone, to afford the title compound as a white solid, 1.44 g.

¹H-nmr (DMSOd₆, 400 MHz) δ: 0.68 (m, 2H), 0.95 (m, 2H), 3.40 (m, 1H), 3.76 (s, 3H), 6.18 (s, 1H), 6.42 (s, 1H).

LRMS: m/z (ES⁻) 231 [M−H⁻]

### Preparation 7

### 2,4-Dichloro-5-cyclopropyl-7-quinazolinyl methyl ether

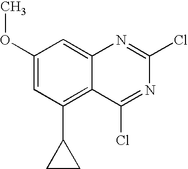

To a solution of the compound from preparation 6 (3.87 g, 16.7 mmol) in phosphorous oxychloride (50 mL) was added N,N-diisopropylethylamine (5.17 g, 40 mmol). The reaction mixture was heated at 100° C. for 1 hour, at reflux for 6 hours then cooled to room temperature. The phosphorous oxychloride was removed under reduced pressure. The resultant oil was partitioned between ethyl acetate (500 mL) and ice-water (300 mL), the layers were separated, the organic phase washed with 1M hydrochloric acid (100 mL), brine (100 mL), dried (MgSO₄) and concentrated under reduced pressure. The residue was purified by column chromatography on silica gel eluting with a solvent gradient of dichloromethane:ethyl acetate (100:0 to 94:6) to give the title compound as a white solid (3.97 g, 88%).

¹H-nmr (CDCl₃, 400 MHz) δ: 0.84 (m, 2H), 1.17 (m, 2H), 2.70 (m, 1H), 2.94 (s, 3H), 7.10 (s, 1H), 7.14 (s, 1H).

LRMS: m/z (ES⁺) 291 [MNa⁺]

### Preparation 8

### 2-Chloro-5-cyclopropyl-7-methoxy-4(3H)-quinazolinone

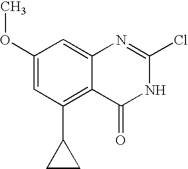

1N Sodium hydroxide solution (30 mL) was added to a solution of the chloro compound from preparation 7 (2.2 g, 8.18 mmol) in dioxane (50 mL) and the reaction stirred at room temperature for 2 hours. The reaction was acidified using 2M hydrochloric acid and extracted with dichloromethane:methanol (95:5) (3×150 mL). The combined organic solutions were dried (MgSO₄) and evaporated under reduced pressure to give the title compound as a white solid, 1.85 g.

¹H-nmr (CDCl₃, 400 MHz) δ: 0.73 (m, 2H), 1.09 (m, 2H), 3.31 (m, 1H), 3.86 (s, 3H), 6.60 (d, 1H), 6.89 (d, 1H).

LRMS: m/z (ES⁺) 273 [MNa⁺]

### Preparation 9

### 1-Benzyl-4-(1-pyrrolidinyl)-1,2, 3,6-tetrahydropyridine

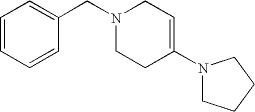

Pyrrolidine (31.8 mL, 0.38 mol) was added to a solution of 1-benzyl-4-piperidinone (48.0 g, 0.25 mol) in toluene (180 mL) and the mixture refluxed under Dean-Stark conditions for 4.5 hours. The reaction mixture was allowed to cool to room temperature and concentrated under reduced pressure to give the title compound as an orange oil (61.8 g, 100%).

¹H-nmr (400 MHz, CDCl₃) δ: 1.80-1.84 (m, 4H), 2.32 (m, 2H), 2.59 (t, 2H), 3.02 (4Hm,), 3.07 (s, 2H), 3.57 (s, 2H), 4.18 (s, 1H), 7.22-7.30 (m, 3H), 7.35-7.36 (d, 2H).

### Preparation 10

### 6-Benzyl-5,6,7,8-tetrahydro[1,6]naphthyridin-2(1H)-one

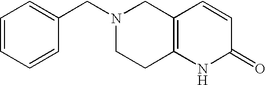

A mixture of the compound from preparation 9 (61.50 g, 0.25 mol) and propiolamide (J. Am. Chem. Soc. 1988; 110; 3968) (35.05 g, 0.51 mol) were heated under reflux in toluene (500 mL) under nitrogen gas for 16 hours. The reaction mixture was allowed to cool to room temperature and concentrated under reduced pressure. The residue was partitioned between dichloromethane (800 mL) and saturated sodium bicarbonate solution (400 mL). The aqueous phase was further extracted with dichloromethane (3×500 mL). The combined organic solutions were dried (MgSO₄) and concentrated under reduced pressure. The residue was purified by column chromatography on silica gel eluting with a solvent gradient of dichloromethane:methanol (100:0 to 95:05) to give the title compound as an orange solid (27.71 g, 45%).

¹H-nmr (DMSOd₆, 400 MHz) δ: 2.53 (t, 2H), 2.63 (t, 2H), 3.24 (s, 2H), 3.60 (s, 2H), 6.06 (d, 1H), 7.08 (d, 1H), 7.24 (m, 1H), 7.30 (m, 4H).

LRMS: m/z (ES⁺) 263 [MNa⁺].

### Preparation 11

### 6-Benzyl-2-bromo-5,6, 7,8-tetrahydro[1,6]naphthyridine

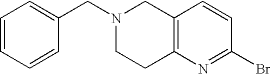

The compound from preparation 10 (9.51 g, 31 mmol) was suspended in acetonitrile (45 mL) and anisole (45 mL). Phosphorous oxybromide (44.8 g, 156 mmol) was added portionwise and the mixture heated for 1 hour at 120° C. The reaction was allowed to cool to room temperature and then poured onto ice (400 g). Dichloromethane (400 mL) was added and the mixture was then neutralised with saturated sodium carbonate solution (450 mL). The organic layer was collected and the aqueous layer extracted with dichloromethane (500 mL). The combined organic solutions were dried (MgSO₄) and concentrated under reduced pressure. The residue was purified by column chromatography on silica gel eluting with a solvent gradient of dichloromethane:methanol (100:0 to 97:03) to give the title compound as a brown oil (5.79 g, 61%).

¹H-nmr (CDCl₃, 400 MHz) δ: 2.83 (t, 2H), 3.03 (t, 2H), 3.56 (s, 2H), 3.70 (s, 2H), 7.12 (d, 1H), 7.21 (d, 1H), 7.26-7.36 (m, 5H).

LRMS: m/z (ES⁺) 303 [MH⁺].

### Preparation 12a

### 6-Benzyl-5,6,7,8-tetrahydro[1,6]naphthyridine-2-carbaldehyde

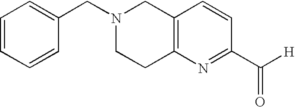

The bromide from preparation 11 (3.00 g, 9.90 mmol) was dissolved in tetrahydofuran (70 mL) and cooled to −78° C. n-Butyl lithium (5.5 mL as a 2.5M solution in hexanes, 13.8 mmol) was added and the reaction stirred for a 5 minutes. N,N-Dimethylformamide (2.3 mL, 29.7 mmol) was then added and the reaction stirred for 1 hour, the cooling bath removed and the reaction quenched by the addition of saturated potassium dihydrogenphosphate solution (100 mL). The residue was purified by flash chromatography on silica gel eluting with dichloromethane:methanol (98:2) to give the title compound as a tan solid (2.10 g, 85%).

¹H-nmr (DMSOd₆, 400 MHz) δ: 2.92 (m, 2H), 3.15 (m, 2H), 3.71 (s, 2H), 3.75 (s, 2H), 7.26-7.38 (m, 5H), 7.45 (d, 1H), 7.73 (d, 1H), 10.02 (s, 1H).

LRMS: m/z (ES⁺) 275 [MNa⁺].

### Preparation 13

### tert-Butyl (2E)-3-(6-benzyl-5,6,7,8-tetrahydro[1,6]naphthyridin-2-yl)-2-propenoate

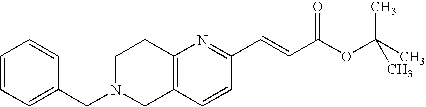

Tri-tert-butylphosphine (3.0 g, 15.28 mmol) was added to a solution of tris(dibenzylideneacetone)dipalladium (4.2 g, 4.63 mmol) in 1,4-dioxane (45 ml), under argon, and the solution stirred for 30 minutes at room temperature. This solution was then added to a mixture of 6-benzyl-2-chloro-5,6,7,8-tetrahydro[1,6]naphthyridine (WO 9830560 Example 33b) (12 g, 46.3 mmol) and tert-butylacrylate (20.3 ml, 139 mmol) in triethylamine (45 ml), and the reaction was stirred under reflux for 17 hours. The cooled mixture was concentrated under reduced pressure and the residue partitioned between ethyl acetate (300 ml) and water (300 ml) and this mixture filtered through Arbocel®. The pH of the mixture was adjusted to 8 using sodium bicarbonate, the phases separated, and the aqueous layer re-extracted with ethyl acetate (2×100 ml). The combined organic solutions were dried over magnesium sulphate and evaporated under reduced pressure. The crude product was pre-adsorbed onto silica gel, and purified by column chromatography using an elution gradient of cyclohexane:ethyl acetate (84:16 to 66:34) to afford the title compound as an orange-red oil, (15.8 g).

¹H-nmr (CDCl₃, 400 MHz) δ: 1.50 (s, 9H), 2.82 (t, 2H), 3.02 (t, 2H), 3.61 (s, 2H), 3.70 (s, 2H), 6.75 (d, 1H), 7.18 (d, 1H), 7.26 (m, 2H), 7.35 (m, 4H), 7.55 (d, 1H).

LRMS: m/z (ES⁺) 373 [MNa⁺]

### Preparation 14

### tert-Butyl (2R,3R)-3-(6-benzyl-5,6,7,8-tetrahydro[1,6]naphthyridin-2-yl)-2,3-dihydroxypropanoate

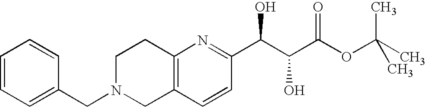

Osmium tetroxide (8.3 ml, 2.5% wt. in tert-butanol) was added dropwise to a mixture of the compound from preparation 13 (11.3 g, 32.2 mmol), N-methylmorpholine N-oxide (4.15 g, 35.4 mmol) in water (80 ml) and acetone (160 ml), and the reaction was stirred at room temperature for 72 hours. The mixture was concentrated under reduced pressure, and the residue azeotroped with acetone. The crude product was purified by column chromatography on silica gel using an elution gradient of cyclohexane:ethyl acetate (80:25 to 25:75), to afford the title compound as a gum (7.2 g).

¹H-nmr (DMSOd₆, 400 MHz) δ: 1.38 (s, 9H), 2.77 (m, 2H), 2.81 (m, 2H), 3.52 (s, 2H), 3.62 (s, 2H), 4.20 (d, 1H), 4.78 (d, 1H), 4.82 (d, 1H), 5.40 (d, 1H), 7.22 (m, 2H), 7.30 (m, 4H), 7.38 (d, 1H).

LRMS: m/z (ES⁺) 407 [MNa⁺]

### Preparation 12b

### 6-Benzyl-5,6,7,8-tetrahydro[1,6]naphthyridine-2-carbaldehyde

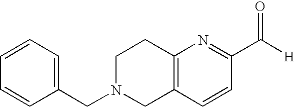

A solution of sodium periodate (4.38 g, 20.5 mmol) in water (38 ml) was added dropwise to a solution of the diol from preparation 14 (7.2 g, 18.7 mmol) in acetonitrile (200 ml), and the reaction was stirred at room temperature for 2 hours. The mixture was partitioned between ethyl acetate (300 ml) and water (300 ml), containing a small volume of brine, and the layers separated. The aqueous phase was further extracted with ethyl acetate (2×100 ml), and the combined organic solutions dried over magnesium sulphate and concentrated under reduced pressure, co-evaporating with tetrahydrofuran. The residual oil was purified by column chromatography on silica gel using an elution gradient of dichloromethane: ethyl acetate (80:20 to 50:50) to afford the title compound, as an oil that crystallised on standing (1.3 g).

¹H-nmr (CDCl₃, 400 MHz) δ: 2.90 (t, 2H), 3.16 (t, 2H), 3.69 (s, 2H), 3.74 (s, 2H), 7.23-7.39 (m, 5H), 7.42 (d, 1H), 7.72 (d, 1H), 10.00 (s, 1H).

LRMS: m/z (ES⁺) 275 [MNa⁺]

### Preparation 15

### N-[(6-benzyl-5,6,7,8-tetrahydro-1,6-naphthyridin-2-yl)methyl]-N,N-dimethylamine

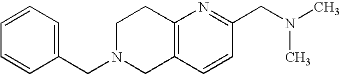

Acetic acid (2.5-3.5eq) followed by dimethylamine (1.1-1.7 eq) were added to a solution of the aldehyde from preparation 12 (1 eq) in tetrahydrofuran (5 mL per mmol) and the solution stirred for 15 minutes. Sodium triacetoxyborohydride (2-2.3 eq.) was added, and the reaction stirred at room temperature for 17 hours. 2N hydrochloric acid was added, to give a pH of 1, the mixture stirred for 15 minutes, then re-basified to pH 12 using 2N sodium hydroxide solution. The mixture was extracted with dichloromethane, the combined organic extracts dried (MgSO₄) and evaporated under reduced pressure. The crude product was purified by column chromatography on silica gel using an elution gradient of dichloromethane:methanol:0.88 ammonia (95:5:0.5 to 90:10:1) to afford the title compound. ¹H-nmr (CDCl₃, 400 MHz) δ: 2.25 (s, 6H), 2.83 (t, 2H), 3.01 (t, 2H), 3.54 (s, 2H), 3.60 (s, 2H), 3.70 (s, 2H), 7.15 (d, 1H), 7.23 (m, 2H), 7.35 (m, 4H).

LRMS: m/z (ES⁺) 304 [MNa⁺]

### Preparation 16

### N,N-Dimethyl(5,6,7,8-tetrahydro[1,6]naphthyridin-2-yl)methanamine

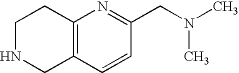

A solution of the protected naphthyridine from preparation 15 (600 mg, 2.13 mmol) in methanol (60 mL) was purged with argon, then heated to reflux. Immediately this was achieved, 10% palladium on charcoal (600 mg) and ammonium formate (268 mg, 4.26 mmol) were added, and the mixture stirred under reflux for 3 minutes. The reaction vessel was then immersed in cold water, and the cooled mixture then filtered through Arbocel®, washing through with ethanol. The filtrate was evaporated under reduced pressure and the residual oil was purified by column chromatography on silica gel using an elution gradient of dichloromethane:methanol:0.88 ammonia (97:3:0.2 to 90:10:1) to afford the title compound as a colourless oil, 259 mg.

¹H-nmr (CDCl₃, 400 MHz) δ: 2.28 (s, 6H), 2.96 (t, 2H), 3.21 (t, 2H), 3.56 (s, 2H), 3.98 (s, 2H), 7.18 (d, 1H), 7.25 (d, 1H). LRMS: m/z (ES⁺) 214 [MNa⁺].

### Preparation 17

### 2-(morpholin-4-ylmethyl)-5,6,7,8-tetrahydro-1,6-naphthyridine

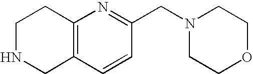

The title compound was prepared from the corresponding protected naphthryidines, following a similar procedure to that described in preparation 16. The title compound was isolated as a yellow oil. ¹H-nmr (CDCl₃, 400 MHz) δ: 2.55 (m, 4H), 2.93 (t, 2H), 3.21 (t, 2H), 3.60 (s, 2H), 3.71 (m, 4H), 3.98 (s, 2H), 7.19 (d, 1H), 7.26 (d, 1H). LRMS: m/z (ES⁺) 234 [MH⁺]

### Preparation 18

### 5-Cyclopropyl-7-methoxy-2-(2-morpholin-4-ylmethyl-7,8-dihydro[1,6]-naphthyridin-6(5H)-yl)-4(3H)-quinazolinone

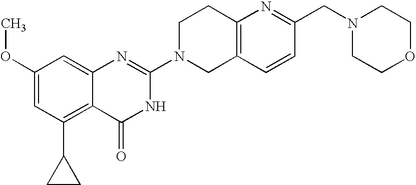

A mixture of the chloride from preparation 8 (351 mg, 1.4 mmol) in n-butanol (21 mL), the amine from preparation 17 (335 mg, 1.43 mmol) and N,N-diisopropylethylamine (633 mg, 4.9 mmol) was heated under reflux for 6 hours, then a further 7 hours at room temperature. The resulting precipitate was filtered off, washed with n-butanol, and dried in vacuo. The solid was purified by column chromatography on silica gel using an elution gradient of dichloromethane:methanol (97:3 to 93:7), and the product triturated with ether to afford the title compound as a white solid, 470 mg.

¹H-nmr (DMSO-d₆, 400 MHz) δ: 0.65 (m, 2H), 0.91 (m, 2H), 2.38 (m, 4H), 2.93 (t, 2H), 3.51 (m, 3H), 3.55 (m, 4H), 3.76 (s, 3H), 3.92 (t, 2H), 4.77 (s, 2H), 6.14 (s, 1H), 6.52 (s, 1H), 7.27 (d, 1H), 7.56 (d, 1H), 11.58 (bs, 1H). LRMS:m/z (ES⁺) 470 [MNa⁺].

Microanalysis found: C, 66.29; H, 6.50; N, 15.48. C₂₅H₂₉N₅O₃;0.3H₂O requires C, 66.29; H, 6.59; N, 15.46%.

1. A method of treating BPH in a subject in need of such treatment which method comprises administering to said subject an effective amount of a combination of (A) atorvastatin or a pharmaceutically acceptable derivative thereof and (B) an α₁-adrenergic receptor antagonist, or a pharmaceutically acceptable derivative thereof.

3. The method of claim 1 wherein (A) and (B) are administered simultaneously, sequentially or separately.

4. The method of claim 1, wherein (B) is selected from terazosin, doxazosin, prazosin, bunazosin, alfuzosin, naftopidil, tamsulosin, silodosin, 4-amino-6,7-dimethoxy-2-(5-methanesulfonamido-1,2, 3,4-tetrahydroisoquinol-2-yl)-5-(2-pyridyl)quinazoline, 5-cyclopropyl-7-methoxy-2-(2-morpholin-4-ylmethyl-7,8-dihydro[1,6]-naphthyridin-6(5H)-yl)-4(3H)-quinazolinone, or a pharmaceutically acceptable derivative thereof.

5. The method of claim 4, wherein (B) is doxazosin or a pharmaceutically acceptable derivative thereof.

6. The method of claim 4, wherein (B) is 4-amino-6,7-dimethoxy-2-(5-methanesulfonamido-1,2, 3,4-tetrahydroisoquinol-2-yl)-5-(2-pyridyl)quinazoline or a pharmaceutically acceptable derivative thereof.

7. The method of claim 4, wherein (B) is 5-cyclopropyl-7-methoxy-2-(2-morpholin-4-ylmethyl-7,8-dihydro[1,6]-naphthyridin-6(5H)-yl)-4(3H)quinazolinone, or a pharmaceutically acceptable derivative thereof.

8. A pharmaceutical composition comprising (A) atorvastatin, or a pharmaceutically acceptable derivative thereof, and (B) doxazosin, or a pharmaceutically acceptable derivative thereof.

9. A pharmaceutical composition comprising (A) atorvastatin or a pharmaceutically acceptable derivative thereof and (B) 4-amino-6,7-dimethoxy-2-(5-methanesulfonamido-1,2,3,4-tetrahydroisoquinol-2-yl)-5-(2-pyridyl)quinazoline or a pharmaceutically acceptable derivative thereof.

10. A pharmaceutical composition comprising (A) atorvastatin or a pharmaceutically acceptable derivative thereof and (B) 5-cyclopropyl-7-methoxy-2-(2-morpholin-4-ylmethyl-7,8-dihydro[1,6]-naphthyridin-6(5H)-yl)-4(3H)-quinazolinone or a pharmaceutically acceptable derivative thereof.

11. A pharmaceutical composition comprising, separately or together, (A) atorvastatin or a pharmaceutically acceptable derivative thereof and (B) doxazosin or a pharmaceutically acceptable derivative thereof, for simultaneous, sequential or separate administration in the treatment of BPH.

12. A pharmaceutical composition comprising, separately or together, (A) atorvastatin or a pharmaceutically acceptable derivative thereof and (B) 4-amino-6,7-dimethoxy-2-(5-methanesulfonamido-1,2,3,4-tetrahydroisoquinol-2-yl)-5-(2-pyridyl)quinazoline or a pharmaceutically acceptable derivative thereof, for simultaneous, sequential or separate administration in the treatment of BPH.

13. A pharmaceutical composition comprising, separately or together, (A) atorvastatin or a pharmaceutically acceptable derivative thereof and (B) 5-cyclopropyl-7-methoxy-2-(2-morpholin-4-ylmethyl-7,8-dihydro[1,6]-naphthyridin-6(5H)-yl)-4(3H)-quinazolinone or a pharmaceutically acceptable derivative thereof, for simultaneous, sequential or separate administration in the treatment of BPH.

14. A pharmaceutical composition according to any one of claims 8 to 13, further comprising a pharmaceutically acceptable carrier.
